(19) United States
(12) Plant Patent Application Publication
(10) Pub. No.: US 2017/0071113 P1
(43) Pub. Date: Mar. 9, 2017

(54) Osteospermum plant name 'Fidosmasuwhimp'

(50) ;

(71) Applicant: Eveline Barends, De Lier (NL)

(72) Inventor: Eveline Barends, De Lier (NL)

(73) Assignee: FIDES B.V., De Lier (NL)

(21) Appl. No.: 14/756,444

(22) Filed: Sep. 4, 2015

(51) Int. Cl. A01H 5/00

(52) U.S. Cl. PLT/360

(57) ABSTRACT

A new and distinct cultivar of Osteospermum plant named ‘Fidosmasuwhimp’, characterized by its uniform and mounding plant habit; vigorous growth habit; freely and uniformly branching habit; freely and early flowering habit; single-type inflorescences with white-colored ray florets; and good garden performance.

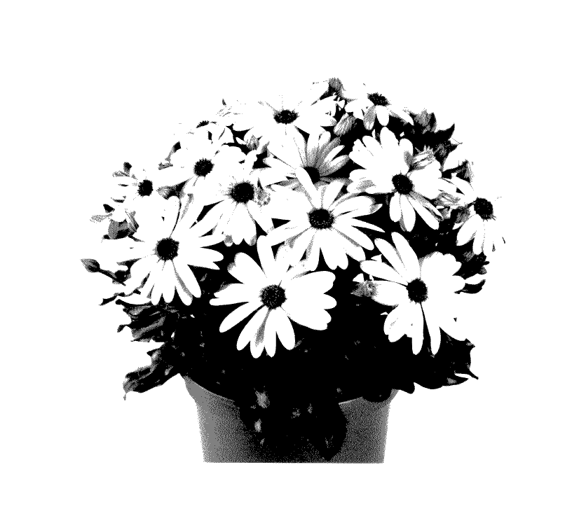

Botanical designation: Osteospermum ecklonis.

Cultivar denomination: ‘FIDOSMASUWHIMP’

### BACKGROUND OF THE INVENTION

The present invention relates to a new and distinct cultivar of Osteospermum plant, botanically known as Osteospermum ecklonis, and hereinafter referred to by the name ‘Fidosmasuwhimp’.

The new Osteospermum plant is a product of a planned breeding program conducted by the Inventor in De Lier, The Netherlands. The objective of the program is to create and develop new vigorous Osteospermum plants that are freely and uniformly branching with numerous large inflorescences with unique and attractive ray and disc floret coloration.

The new Osteospermum plant originated from a cross-pollination by the Inventor in August, 2008 of a proprietary selection of Osteospermum ecklonis identified as code number O 45719, not patented, as the female, or seed, parent with a proprietary selection of Osteospermum ecklonis identified as code number O 67250, not patented, as the male, or pollen, parent. The new Osteospermum plant was discovered and selected by the Inventor as a single flowering plant within the progeny of the stated cross-pollination in a controlled greenhouse environment in De Lier, The Netherlands in July, 2009.

Asexual reproduction of the new Osteospermum plant by terminal vegetative cuttings in a controlled greenhouse environment in De Lier, The Netherlands since July, 2009 has shown that the unique features of this new Osteospermum plant are stable and reproduced true to type in successive generations.

### SUMMARY OF THE INVENTION

Plants of the new Osteospermum have not been observed under all possible combinations of environmental conditions and cultural practices. The phenotype may vary somewhat with variations in environmental conditions such as temperature and light intensity, without, however, any variance in genotype.

The following traits have been repeatedly observed and are determined to be the unique characteristics of ‘Fidosmasuwhimp’. These characteristics in combination distinguish ‘Fidosmasuwhimp’ as a new and distinct Osteospermum plant:

1. Uniform and mounding plant habit.

2. Vigorous growth habit.

3. Freely and uniformly branching habit.

4. Freely and early flowering habit.

5. Single-type inflorescences with white-colored ray florets.

6. Good garden performance.

Plants of the new Osteospermum differ primarily from plants of the parent selections in ray floret color as plants of the female parent selection have purple-colored ray florets and plants of the male parent selection have pink-colored ray florets.

Plants of the new Osteospermum can be compared to plants of the Osteospermum ecklonis ‘Fidostsupwhi’, disclosed in U.S. Plant Pat. No. 25,081. In side-by-side comparisons conducted in De Lier, The Netherlands, plants of the new Osteospermum differed primarily from plants of ‘Fidostsupwhi’ in the following characteristics:

1. Plants of the new Osteospermum were more vigorous than plants of ‘Fidostsupwhi’.

2. Plants of the new Osteospermum had larger inflorescences than plants of ‘Fidostsupwhi’.

Plants of the new Osteospermum can be compared to plants of the Osteospermum ecklonis ‘Duetiswewi’, disclosed in U.S. Plant Pat. No. 24,994. In side-by-side comparisons conducted in De Lier, The Netherlands, plants of the new Osteospermum differed primarily from plants of ‘Duetiswewi’ in the following characteristics:

1. Plants of the new Osteospermum were more freely branching than plants of ‘Duetiswewi’.

2. Plants of the new Osteospermum had larger inflorescences than plants of ‘Duetiswewi’.

### BRIEF DESCRIPTION OF THE PHOTOGRAPH

The accompanying photograph illustrates the overall appearance of the new Osteospermum plant showing the colors as true as it is reasonably possible to obtain in colored reproductions of this type. Colors in the photograph may differ slightly from the color values cited in the detailed botanical description which accurately describe the colors of the new Osteospermum plant. The photograph is a side perspective view of a typical flowering plant of ‘Fidosmasuwhimp’ grown in a container.

### DETAILED BOTANICAL DESCRIPTION

The aforementioned photograph, following observations and measurements describe plants grown during the summer in 10.5-cm containers in a glass-covered greenhouse in De Lier, The Netherlands and under cultural practices typical of commercial Osteospermum production. During the production of the plants, day and night temperatures averaged 18° C. and light levels averaged 4,500 lux. Plants were pinched three weeks after planting and were 13 weeks old when the photograph and description were taken. In the following description, color references are made to The Royal Horticultural Society Colour Chart, 2007 Edition, except where general terms of ordinary dictionary significance are used.
Botanical classification: Osteospermum ecklonis ‘Fidosmasuwhimp’.
Parentage:
   Female, or seed, parent.—Proprietary selection of Osteospermum ecklonis identified as code number O 45719, not patented.
   Male, or pollen, parent.—Proprietary selection of Osteospermum ecklonis identified as code number O 67250, not patented.
Propagation:
   Type.—Terminal vegetative cuttings.
   Time to initiate roots, summer.—About five days at temperatures about 20° C.
   Time to initiate roots, winter.—About seven days at temperatures about 20° C.
   Time to produce a rooted cutting, summer.—About three weeks at temperatures about 20° C.
   Time to produce a rooted cutting, winter.—About four weeks at temperatures about 20° C.
   Root description.—Fine, fibrous; typically white in color, actual color of the roots is dependent on substrate composition, water quality, fertilizers, substrate temperature and age of roots.
   Rooting habit.—Freely branching; medium density.
Plant description:
   Plant form and growth habit.—Uniform and mounding plant habit; relatively short internodes, dense and bushy growth habit; vigorous growth habit.
   Plant height.—About 34 cm.
   Plant diameter.—About 58 cm.
   Lateral branches.—Quantity per plant: Freely branching habit, about six lateral branches developing per plant; pinching enhances lateral branch development. Length: About 38 cm. Diameter: About 6 mm. Internode length: About 1.2 cm. Strength: Strong, sturdy. Texture: Smooth, glabrous. Color: Close to 144B.
   Leaf description.—Arrangement: Alternate, simple; sessile. Length: About 7.8 cm. Width: About 2.9 cm. Shape: Spatulate. Apex: Acute. Base: Attenuate. Margin: Dentate; sinuses divergent. Texture, upper surface: Pubescent; leathery. Texture, lower surface: Smooth, glabrous. Venation pattern: Pinnate. Color: Developing leaves, upper surface: Close to 137B. Developing leaves, lower surface: Close to 144A. Fully expanded leaves, upper surface: Close to 137A; venation, close to 144B. Fully expanded leaves, lower surface: Close to 137B to 137C; venation, close to 144B.
Inflorescence description:
   Appearance and aspect.—Terminal and axillary inflorescences; inflorescences positioned beyond the foliar plane on strong peduncles and face mostly upright to outwardly; single-type inflorescence form with lanceolate-shaped ray florets and tubular disc florets; ray and disc florets developing acropetally on a capitulum.
   Flowering habit.—Freely flowering habit; about 80 inflorescences developing per plant.
   Fragrance.—None detected.
   Flowering response.—In The Netherlands, plants of the new Osteospermum flower continuously from spring until first frost in the autumn; early flowering habit, plants begin flowering about six weeks after planting.
   Inflorescence longevity.—Inflorescences last about three weeks on the plant; inflorescences not persistent.
   Inflorescence buds, just prior to anthesis.—Height: About 1.5 cm. Diameter: About 1.2 cm. Shape: Ovoid. Color: Close to 137B and 144C.
   Inflorescence size.—Diameter: About 8.2 cm. Depth (height): About 2.5 cm. Disc diameter: About 1.2 cm. Receptacle diameter: About 1 cm. Receptacle height: About 3 mm.
   Ray florets.—Length: About 4.1 cm. Width: About 1 cm. Shape: Lanceolate. Apex: Obtuse to emarginate. Base: Attenuate. Margin: Entire. Texture, upper and lower surfaces: Smooth, glabrous. Luster, upper and lower surfaces: Satiny. Quantity per inflorescence and arrangement: About 16 arranged in one to two whorls. Color: When opening and fully opened, upper surface: Close to N155A; color does not change with development. When opening and fully opened, lower surface: Close to N155A; longitudinal stripes, close to N81B; color does not change with development.
   Disc florets.—Shape: Tubular with five pointed apices. Length: About 5 mm. Diameter: About 1 mm. Number of disc florets per inflorescence: About 50. Color, immature: Close to 93C. Color, mature: Close to 93A to 93C.
   Phyllaries.—Quantity per inflorescence and arrangement: About 16 arranged in one to two whorls. Length: About 1 cm. Width: About 1.6 mm. Shape: Lanceolate. Apex: Apiculate. Base: Attenuate. Margin: Entire. Texture, upper and lower surfaces: Smooth, glabrous; leathery. Luster, upper and lower surfaces: Matte. Color, upper surface: Close to 137B to 137C. Color, lower surface: Close to 137C.
   Peduncles.—Length: About 22 cm. Diameter: About 2 mm. Strength: Strong. Aspect: Mostly upright to outwardly slanting. Texture: Smooth, glabrous. Color: Close to 144A.
   Reproductive organs.—Androecium: Present on disc florets only. Anther shape: Lanceolate. Anther length: About 2 mm. Anther color: Close to 79A to 79B. Pollen amount: Abundant. Pollen color: Close to 23A. Gynoecium: Present on both ray and disc florets. Pistil length: About 7 mm. Stigma shape: Bi-parted. Stigma color: Close to 79A. Style length: About 3 mm. Style color: Close to 79A. Ovary color: Close to 144C.
   Seeds.—Length: About 7 mm. Diameter: About 3 mm. Texture: Smooth, glabrous. Color: Close to 199A.
Disease & pest resistance: Plants of the new Osteospermum have not been observed to be resistant to pathogens and pests common to Osteospermum plants.
Garden performance: Plants of the new Osteospermum have been observed to have good garden performance and to tolerate rain, wind and temperatures ranging from about 5° C. to about 35° C.

It is claimed:

1. A new and distinct Osteospermum plant named ‘Fidosmasuwhimp’ as illustrated and described.
